(19) United States
(12) Patent Application Publication
(10) Pub. No.: US 2026/0007806 A1
(43) Pub. Date: Jan. 8, 2026

(54) 3D PRINTED BIOACTIVE SCAFFOLDS

(71) Applicant: GENIS HF., Siglufjordur (IS)

(72) Inventors: Chuen How NG, Gardabaer (IS); Gissur ORLYGSSON, Mosfellsbaer (IS)

(21) Appl. No.: 19/099,817

(22) PCT Filed: Aug. 11, 2023
(86) PCT No.: PCT/IS2023/060002

§ 371 Date: Jan. 30, 2025

(30) Foreign Application Priority Data
Aug. 11, 2022 (IS) 050374

(51) Int. Cl. A61L 27/48; A61L 27/20

(52) U.S. Cl. CPC A61L 27/48; A61L 27/20; A61L 2430/02

(57) ABSTRACT

Provided is an implantable tissue scaffold comprising a mixture of a biocompatible organic polymer and chitin, wherein the chitin is embedded in the biocompatible organic polymer. Also provided is a composition for 3D printing, the composition comprising at least one biocompatible organic polymer and chitin that may be partially deacetylated, wherein the chitin is embedded within the biocompatible organic polymer. Further provided is a method of promoting tissue formation, comprising implanting a tissue scaffold comprising a mixture of a biocompatible organic polymer and chitin at a site in need of regenerative bone tissue formation.

### FIELD

The invention is within the field of medical implants, in particular implants comprising bioactive and biocompatible materials that are suitable for use in osteoinductive and/or osteogenic applications. The invention is also within the field of 3D printable compositions, in particular compositions comprising bioactive and/or biocompatible organic polymers.

### INTRODUCTION

Medical knowledge on trauma and other bone defect management has greatly advanced and improved throughout the world. However, the gold standard for restoring bone defects is still considered to be autologous bone grafting. Clinical benefits are not guaranteed and donor-site complications and morbidity are not infrequent. Occasionally, one or several additional interventions are needed and the transplant material is limited. Until now, segmental bone defects caused by trauma, bone tumors, revision surgeries or infection are still a great challenge for trauma surgeons to overcome. Although several materials and various implant options have been developed or improved, the perfect solution, especially the filling up of critical size defects, still remains to be explored.

Tissue engineering is the remodeling or reconstruction of human tissue through artificial manipulation, deliberately guiding tissue growth through controlled molecular signals and/or designated physical/mechanical channeling. Presently, the preparation of three dimensional scaffolds is dominated by conventional manufacturing techniques, including phase separation, solvent casting, membrane coating, electrospinning, molding and foaming. However, all these methods share a major drawback in that they do not permit full control of the architecture of the scaffold, its pore network and pore size, giving rise to inconsistent and less-than-ideal scaffolds.

3D printing technologies are becoming more and more critical in tissue engineering as they possess the capability of integrating biomedical device designs into the practical fabrication process, thereby providing opportunities to fabricate objects with both controlled macro and microarchitecture structures. Compared to conventional material processing techniques for tissue engineering of highly complex tissues like bone, 3D printing technologies offer much more precise, reproducible fine details, as well as providing a more systematic approach for further research and development. In real-time conditions, the high reproducibility of an instrumental process will be an aid to surgeons and minimize the potential errors of a surgical process, since the methodology provides opportunities for designing and printing a customized scaffold during an operation procedure, based on real-time assessment and imaging of the required implant.

Apart from being printable and osteoinductive/osteoconductive, suitable material for tissue engineering, in particular bone regeneration applications, should be biodegradable, antimicrobial, endotoxin-free, non-toxic and mechanically stable. Numerous materials have been reported on for bone scaffolds, including collagen, hyaluronic acid, hydroxyapatite, bioglass, titanium, polylactic acid, PMMA, carbon nanotubes, etc. However, it emerges that all these materials are osteoconductive, but lack osteoinductive properties, and therefore do not meet the required osteoinductive/osteoconductive demands of implants (Baldwin et al., 2019, J Orthopaed Trauma 33:203; D'Souza et al., 2019, Biomedicines 7:1). In this context, there therefore is an urgent demand for 3D printable osteoinductive and osteoconductive biomaterials to be developed to fulfill near future orthopedic application needs.

### SUMMARY

The present invention seeks to overcome the above mentioned deficiencies and shortcomings of the prior art. The objective of the disclosure is to provide tissue scaffolds, compositions for preparing such scaffolds and methods where bioregenerative properties, in particular properties characterized by consistent osteoinductive/osteoconductive properties, are combined with printable solutions, in particular 3D printing. A key feature is the incorporation of non-conventional chitin material into a biocompatible and/or biodegradable osteoconductive polymer to form a composite material that is suitable for use with additive manufacturing methods based on e.g. material extrusion.

Thus, in one aspect there is provided an implantable tissue scaffold comprising a mixture of a biocompatible organic polymer and chitin, wherein the chitin is physically embedded in the biocompatible organic polymer.

The chitin used in the scaffolds and compositions described herein is a partially deacetylated chitin material with controlled distribution of glucosamine moieties in the polymeric chain and. The chitin material can be provided as distinct particles that are dispersed within the biocompatible organic polymer. The implantable tissue scaffold can be 3D printed using conventional 3D printing technology.

The implantable tissue scaffold is adapted to be implanted in a human or animal body. Upon implantation, chitin that is embedded within the scaffold will slowly be released and degraded by natural processes, leading to the formation of desired short chain chitooligosaccharides (COS). The thus released COS will promote healing and the formation of natural tissue growth at the site of implantation.

Another aspect relates to a composition for 3D printing, the composition comprising in the range of about 75%-99.95% by weight of at least one biocompatible organic polymer and in the range of about 0.05%-20% chitin. The chitin is preferably embedded within the biocompatible organic polymer in the composition, in the form of distinct particles. The chitin may be partially deacetylated, as described in more detail herein.

It follows that the implantable tissue scaffold can be formed by a process of 3D printing the composition. Another aspect therefore relates to method of preparing a tissue scaffold comprising 3D printing a composition as set forth herein. The 3D printing can be performed based on three dimensional modeling of tissue defects to be repaired and/or healed, such as bone defects, bone fractures and the like. Such modeling can be based on analysis of one or more imaging methods including computerized tomography (CT), magnetic resonance imaging (MRI), radiology bone scan, ultrasound imaging, radionucleotide bone imaging.

Another aspect relates to a method of promoting tissue formation, comprising implanting a tissue scaffold as described herein at a site in need of regenerative tissue formation in a human or animal body. In certain embodiments, the tissue in question is bone tissue. In certain embodiments, the method relates to a human body. In certain embodiments, the method relates to an animal body.

Another aspect relates to a method of treatment of a bone tissue defect, the method comprising implanting a tissue scaffold as described herein at a site in need of regenerative bone tissue formation in a human or animal body.

Yet another aspect relates to an implantable tissue scaffold as described herein for use in the treatment of a tissue defect in a human or animal body. The tissue defect can preferably be a bone tissue defect, such as fractured or missing bone.

The above features along with additional details of the inventions, are described further in the examples below, which are intended to further illustrate the invention but are not intended to limit its scope in any way.

### BRIEF DESCRIPTION OF THE DRAWINGS

The skilled person will understand that the drawings, described below, are for illustration purposes only. The drawings are not intended to limit the scope of the present teaching in any way.

FIG. 1 shows an image obtained by scanning electron microscopy (SEM) of partially deacetylated chitin (PDC) used in the preparation of a tissue scaffold.

FIG. 2 shows an SEM image of a tissue scaffold, visualizing PDC particles (shown in circles) and larger CaP particles (shown in rectangular boxes).

FIG. 3 shows images obtained by micro-CT showing the even distribution of CaP particles within filaments, with cross-sectional views being shown in (a) and longitudinal views in (b) for different composition of CaP/PDC (see Table 2).

FIG. 4 shows results of stability study of partially deacetylated chitin during 3D printing at 200° C. as determined by X-ray diffraction (XRD); (a) stability over time; (b) crystallinity and crystal size for various treatment times.

FIG. 5 shows different types of unit cells used for 3D printing PDC/PLA scaffolds, including straight stacked beams (SSB, A), porous sodalite crystal (PSC, B) and porous hexagonal prism (PHP, C). For each scaffold type, the porosity is varied between 25%, 50% and 70% (left to right for each scaffold shown).

FIG. 6 shows SEM images of 3D printed scaffolds at both macroscopic and microscopic level. For each cell type, macroscopic views are shown on the right, and microscopic views on the left.

FIG. 7 shows results of determination of the compressive strength of 3D printed PDC/PLA scaffolds, containing 0.25%, 0.5% and 1.5% PDC.

FIG. 8 shows compressive strength of 3D printed PDC/PLA scaffolds containing varying amounts of PDC, for two types of structures, straight stacked beams (SSB) and porous sodalite crystal (PSC).

FIG. 9 illustrates the conceptual framework in accordance with the present invention in the context of bone regeneration applications.

FIG. 10 shows CT images of sites of operation in the femur of rats receiving control implant (A) and implant containing 0.25% chitosan (B). FIG. 11 shows results of bone formation determination from CT images of animals included in the rat trial.

FIG. 12 shows in (a) highly swollen partially deacetylated (about 50%) chitin material; (b) a crystal clear solution that forms upon the chitin material being dissolved with 100% solubility.

### DESCRIPTION

The present disclosure provides a conceptual framework of 3D printable biopolymer composite materials that have bioregenerative properties. Such materials are suitable e.g. for use in the production of customized 3D printed scaffolds and material extrusion (fused deposition modeling, FDM). In these composite materials, chitin is incorporated into a biodegradable polymer, providing a biopolymer composite comprising the biodegradable polymer and chitin material. The biodegradable polymer provides mechanical stability and osteoconductive properties, while the chitin is believed to provide osteoinductive properties to the resulting composite material, in addition to the well-known anti-microbial and hemostatic properties of chitosan.

The biopolymer composite can be 3D printed to generate scaffolds of any desirable structure and porosity. Thereby, a designed scaffold can be designed and printed for use in situ, where the scaffold provides mechanical stability at the site of implant. With time, the biodegradable polymer is replaced by naturally formed bone tissue, which generation is aided by the osteoinductive properties of the chitin material in the polymer. New bone tissue migrates into the porous scaffold, where new bone tissue is formed, stimulated by the osteoconductive properties of the scaffold and osteoinductive properties of the chitin material.

Chitin is a linear polysaccharide composed of N-Acetyl Glucosamine (GlcNAc) monosaccharides which are linked by 1-4 β linkage to form a linear biopolymer. Chitin is an essential constituent of the exoskeleton of crustaceans and insects and in some specific organs of mollusks such as the pen of squid and cuttlefish.

The chitin material can be partially or fully deacetylated, wherein 0% deacetylated chitin is a homopolymer of N-acetylglucosamine subunits connected by covalent β-(1→4)-linkages, and 100% deacetylated chitin is a homopolymer of glucosamine (GlcN) subunits connected by covalent B-(1→4)-linkages. Chitosan is the deacetylated form of chitin, containing a mixture of GlcN and GlcNAc.

There are 3 different types of chitin, namely, alpha, beta and gamma chitin. All these chitin are with different crystalline state. This is mainly due to the orientation or the packing of the polymeric chains in the matrix. With this difference, for deacetylated chitin with identical % DD, the physico-chemical properties may be quite different depending on the crystalline state.

On the other hand, there are at least 3 different types of deacetylation methods, including a) solid state or heterogenous deacetylation at high temperature (herein referred to as SST), b) liquid state or homogenous deacetylation at low temperature (herein referred to as LSL), and c) solid state or heterogenous deacetylation at low temperature (herein referred to as SSL). The SST deacetylation method is now commonly adpoted in the chitin industry. Comparing the distribution of glucosamine deacetylation at 50% DD, the SST deacetylation will produce large amount of block or clustered glucosamine in the polymeric chain; the sequence of gluscosamine and n-acetyl-D-glucosamine for the LSL method is generally arranged in alternate manner, i.e. one monomer next to other; and for SSL, it is in between the SST and LSL form, or in a random form. It is due to this distribution pattern of glucosamine or N-acetyl-D-glucosamine, for a similar degree of deacetylation that the physico-chemical properties of the deacetylated material may have complete different characteristics, e.g. one can have highly swollen properties upon contact with water and yet the SST material may have no swelling for the same % DD material; or one can be fully dissolved in acid and other may show little dissolution only. Upon degradation in the body, the difference in the resulting oligomer patterns of these 3 types of deacetylation will even be more significant, leading to different in vivo effects on tissue regeneration.

The degree of deacetylation (% DD) and the average molecular weight are the two most important characteristics taken into consideration in current chitin industry. However, this is referring to chitin with % DD of 75% and higher. For chitin with 75% DD and lower, more characterization is needed to understand its properties, especially the distribution of the glucosamine moieties in the polymeric chain and the crystalline state of the material. Partially deacetylated chitin (chitosan) or chitosan has more complex physio-chemical properties, such that being somewhat or highly crystalline, or alternatively the partially deacetylated chitin can have an amorphous structure. These characteristics are highly dependent on the manufacture process and hence the molecular composition of the partially deacetylated chitin. A further complications is due to the different types of chitin, i.e. the alpha, beta and gamma forms which all have their unique properties.

Thus, for a chitin with a similar degree of deacetylation, different distribution of glucosamine moieties will have great impacts on all their physical properties, including toughness or mechanical properties, swelling, dissolution and thermal properties of the material (Aiba, S. Int J Biol Macromol, 1991, 13 (1), 40-44; Sannan, T., et al., Makromol Chemie, 1976, 177 (12), 3589-3600). As described, the manufacturing process in current chitosan industry is dorminant by less-controlled high temperature process in their production, resulting in inferior chitosan that can be only be applicable less demanded industries, and will not fulfill the demand for medicinal or orthopedic purpores. The material used herein is a result of carefully controlling the distribution of glucosamine to its desired arrangement, to optimize the physico-chemical properties and leading to desired oligosaccharide (COS) pattern after degration in vivo to initiate tissue regeneration effectively. The resulting material preferably has a random distribution of glucosamine (and thereby also a random distribution of N-acetylglucosamine) moieties within the polymeric chain.

The chitin material is preferably manufactured by a particular process to fullfil specific characteristics especially a) the controlled distribution of the glucosamine moieties in the chitin matrix and within the polymeric chain; b) a low amorphous status to ensure a smooth and desired degradation patterns. These two parameters are crucial to the extent that for a chitin with a similar degree of deacetylation, different distribution of glucosamine moieties will have great impact on the physical properties of the different chitin polymers, including toughness or mechanical properties, swelling, dissolution, thermal properties of the material and resistance to precipitation upon dissolution.

Different physiochemical properties of the chitin material also results in different biological response to the material. Thus, for resulting biological impact, upon degredation in the body, different glucosamine distribution will affect enzymatic cutting sites, thereby bringing about different degraded oligomeric products. Since these degradation products are believed to underly the biological activity through binding to chitinase-like proteins (CLP) or other receptors in the human or animal body, the nature and distribution of these products (shorty chain oligosaccharides) results in different biological responses and thus substantial differential impacts on tissue regenetion.

The percentage of the degree of deacetylation (% DD) is the fraction of GlcN in the copolymers comprised of GlcNAc and GlcN. The % DD value of a chitin/chitosan sample is one of the crucial factor in assessing its properties. Chitin having a % DD value less than 35% is insoluble in weak acids, e.g. 1% acetic acid. Chitin/chitosan with % DD higher than 75% is however soluble in weak acids. The solubility of partially deacetylated chitin in weak acid is highly affected by its processing method. A chitin material with a random distribution of glucosamine and a % DD of about 50% is soluble in a weak acid such as acetic acid. Such material is therefore expected to have a higher bioavailability compared with less soluble chitin material.

Chitin that has been partially deacetylated, i.e. chitin that has a degree of deacetylation >0% is sometimes also called chitosan. It follows that chitosan can in general comprise any desired degree of deacetylation. Moreover, the chitosan can be randomly deacetylated, i.e. the deacetylation can be at random positions in the polymeric chain, or the chitosan can be a block deacetylated, i.e. the deacetylation pattern is with clustered or aggregated glucosamine in the polymeric chain. As described herein, the term “chitin” refers to chitin material that can have any degree of deacetylation, i.e. the degree of deacetylation can be anywhere from 0-100%. The term “chitosan”, as described herein, refers to chitin material that is partially (i.e. >0%) deacetylated. It follows that the terms “chitin” and “chitosan” can be used interchangeably for partially deacetylated chitin (PDC) material.

Compared to fully acetylated chitin, partially deacetylated chitooligosaccharides (oligosaccharides) are smaller and water soluble, and are prepared by deacetylating chitin to produce chitosan. Either chitin or chitosan can be hydrolyzed to produce chitooligosaccharides with varying degrees of acetylation. Short-chain chitooligosaccharides are more soluble in water than polymeric chitin; in particular, chitooligosaccharides that are significantly deacetylated are highly water soluble.

The swelling index and the solubility are two easy method to assess of the distribution of glucosamine for chitin with 75% DD and lower. The more random is the distribution of the glucosamine in the polymeric chain, the more swelling and better solubility for a chitin. This is especially prominant in a 50% DD chitin. For example, the swelling index for a chitin film coated with randomly deacetylated 50% DD chitin may reach 6-10 times of its dry weight, and generally with high solubility. Also, the chitin will have a comparatively lower crystalline state for a chitin with more randomly distributed glucosamine. Additionally, a chitin solution prepared will be more resistance to precipitation for a more than a less random distributed ones.

Bone graft materials are typically classified into 3 types, i.e. osteoconductive, osteoinductive and osteogenic. Osteoconduction refers to the grafting of material to serves as a scaffold for new bone growth that is perpetuated by the native bone. The idea is to allow the passive growth of new bone into the scaffold or channel itself into the designated dimensions, e.g. into pores, channels or pipes. Osteoinduction is a process in which osteogenesis is induced, e.g. by a biomaterial. Osteoinduction involves cell recruitment and the activation of these cells to develop into preosteoblasts. It is a regular phenomenon seen in the bone healing process. In a bone fracture situation, the majority of bone heals by the osteoinduction process. Osteogenesis refers to the process in which bone producing cells, either presenting directly (as osteocytes, osteoblasts or chondroblasts) or indirectly from previously undifferentiated stem cells, create new bone by laying down osteoid or via endochondral ossification of cartilage.

Recent research performed by the applicant has shown that chitosan possesses osteoinductive properties, which makes it an ideal candidate as a component in material for bone regeneration (Kjalarsdóttir, et al., 2019, Regen Biomater 6:231). Moreover, the degree of deacetylation of the chitosan and the method of preparing the material play a decisive role in the osteoinductive properties. Thus it was shown that chitin with a degree of deacetylation in the range of about 50% to 70% with this deacetylation method leads to particularly high levels of tissue regeneration than highly deacetylated chitosan.

Chitosan has been shown to offer many advantages in biomedical applications, including biocompatibility and biodegradability in a controlled manner, and modulation of inflammatory responses. Additionally, chitosan, due to its bioresorbable, antimicrobial, nontoxic and polycationic nature, and immense potential in tissue regeneration, has a wide spectrum of applications in the pharmaceutical/medical fields, such as: antimicrobial agent, controlled drug delivery, blood anticoagulant, wound dressing, and tissue engineering, including bone and nerve regeneration. Chitosan has been extensively studied in bone tissue engineering. Chitosan may act alone, or it can be blended with other polymers, with natural and synthetic materials, which is generally considered an effective way to develop tissue-engineering, chitosan-based materials, such as 3D lyophilized scaffolds, hydrogels, films, and other scaffolds.

Chitosan has demonstrated its osteoinductive/osteoconductive properties in many studies (Tan et al. 2014, Biomaterials 35:7828; Geffre et al. 2010, Future Sci OA 4: FSO225). In vivo studies suggest that chitosan alone is sufficient to stimulate osteogenesis [Pang et al., 2017, Oncotargt 8:35583; Ho et al, 2015, Int J Nanomedicine 10:5941). More importantly, it was revealed both in vitro and in vivo that the degree of deacetylation (DD) of the chitosan material has decisive impacts on bone formation (Lieder et al. 2012, J Biomed Mater Res Part A, 100A: 3392; Kjalarsdottir et al. 2019, Regen Biomater 6:231). However, chitosan by itself lacks adequate mechanical strength to be suitable for load-bearing applications.

To overcome this deficiency of chitosan and provide mechanically stable solution, incorporation of chitin (in particular partially deacetylated chitin) into suitable biocompatible and/or biodegradable polymers is provided in the tissue scaffolds and compositions described herein.

In general, the amount of chitin in the tissue scaffolds and/or compositions can be in the range of about 0% to 20%, in the range of about 0% to 10%, in the range of about 0% to 5%, in the range of about 0.05% to 20%, in the range of about 0.05 to 15%, in the range of about 0.05% to 10%, in the range of about 0.05% to 5%, in the range of about 0.05 to 3%, in the range of about 0.1 to 20%, in the range of about 0.2 to 20%, in the range of about 0.5 to 20%, in the range of about 0.7 to 20%, or in the range of about 1%-20%. The lower end of the range can be about 0.05%, about 0.1%, about 0.15%, about 0.2%, about 0.25%, about 0.3%, about 0.4%, about 0.5%, about 0.6%, about 0.7%, about 0.8%, about 0.9% or about 1.0%. The upper end of the range can be about 1%, about 1.5%, about 2.0%, about 2.5%, about 3.0%, about 3.5%, about 4%, about 4.5%, about 5%, about 7%, about 8%, about 10%, about 12%, about 14%, about 15%, about 16%, about 18% or about 20%.

The amount of the chitin material used in the scaffold and/or compositions may depend on its degree of deacetylation (% DD). Thus, PDC material with a relatively low % DD (e.g., less than 75%) may be present in a lower amount in the scaffolds than a PDC material with a relatively high % DD (e.g, higher than 75%). For example, for chitin with % DD higher than 75%, the amount of chitin in the scaffold can be in range of 0.1 to 20%, in range of 0.1 to 10%, or in rnage of 0.1 to 5%.

The chitin material can be partially or fully deacetylated. For example, the chitin material can have a degree of deacetylation in the range of 0 to 100%, in the range about 10% to about 90% (w/w), in the range of about 10% to about 70% (w/w), in the range of about 20% to about 70% (w/w), in the range of about 30% to about 70% (w/w), in the range of 35 to about 65%, in the range about 35 to 60%, in the range of about 40% to about 60% (w/w), in the range about 40 to about 55%, or in the range of about 45% to about 55% (w/w).

The lower end of the range of suitable degree of deacetylation can be 0%, about 5%, about 10%, about 20%, about 30%, about 40%, about 45%, about 50%, about 55%, about 60%, about 65%, or about 70%. The upper end of suitable degree of deacetylation can be about 70%, about 75%, about 80%, about 85%, about 90%, or about 99%.

In certain applications, the chitin material has a degree of deacetylation in the range of about 45% to about 95% (w/w), in the range of about 50% to about 90% (w/w), in the range of about 50% to about 80% (w/w), or in the range of about 50% to about 70% (w/w).

The chitin material can have a weight-average molecular weight (MW) in the range of 200Da-2000 kDa, such as in the range of 1 kDa-1000 kDa, in the range of 5 kDa-500 kDa, in the range of 10 kDa-300 kDa or 20 kDa-1500 kDa.

It can be preferably that the chitin material comprise an amorphous partially deacetylated chitosan (PDC). The material can be produced by a controlled process with specific glucosamine distribution resulting in material that is soluble in an acidic aqueous solution and has a high swelling capability. Such chitosan is preferably about 45% to about 55% deacetylated (such as about 50%), and is completely soluble in an acidic solution (i.e., 100% solubility). The chitosan can have a weight-average molecular weight that is in the range of 100 kDa-400 kDa, such as about 300 kDa. The chitosan can absorb as much as 10x, 15x or about 20x or more of its weight of water.

In some embodiments, the chitin is completely soluble in a weakly acidic solution, such as in an acetic acid solution. In other words, the chitin is 100% or near 100% (such as greater than 99%) soluble in the acidic solution. The chitin material dissolves instantaneously, or near instantaneously (within a few minutes, such as within 10 minutes or within 5 minutes) upon acidification. This is different from chitosan known in the art, which typically dissolves very slowly, or not at all, in acidic solution.

Solubility can be assessed by passing a solution containing the PDC through a filter, such as a 0.45 micrometer filter, with no insoluble particles not passing through the filter being a measure of complete solubility.

The chitin material has a high swelling capability, forming a gel upon contact with water. The ability to form a gel can be manifested by ability to absorb (in a dry state) 10x or more, 15x or more, or 20x or more of the weight of water. The ability to form a gel with swelling of 10x or more is particularly pronounced for chitin with high molecular weight, such as a molecular weight of 150 kDa or more, 200 kDa or more or 250 kDa or more.

The chitin material can preferably be in the form of microparticles. The microparticles can have an average particle diameter in the range of about 0.1 μm to about 50 μm, in the range of about 1 μm to about 25 μm, in the range of about 1 to 15 μm, in the range of 5 to 20 μm, or in the range of about 5 μm to about 15 μm. The lower end of the range can be about 1 μm, about 2 μm, about 3 μm, about 4 μm or about 5 μm. The upper end of the range can be about 10 μm, about 15 μm, about 20 μm, about 25 μm, about 30 μm, about 40 μm or about 50 μm. The microparticles are preferably physically embedded and dispersed within the biocompatible organic polymer. The microparticles can thus be visualized as distinct physical entities within the organic polymer. The particles can be homogeneously dispersed within the organic polymer. By physically embedding the particles within the polymer, solubility problems of the chitin are eliminated. Therefore, physical embedding of chitin within the polymer is suitable for all chitin, independent of its degree of deacetylation. The microparticles can preferably contain only chitin material, i.e. the particles consist essentially only of chitin material. The implantable scaffold thereby comprises a biocompatible and/or biodregadable organic polymer and microparticles consisting essentially of chitin material.

Any suitable organic polymer that is biocompatible and/or biodegradable can be used in the applications described herein. For example, the organic polymer can be selected from polylactic acid (PLA), poly (lactic acid co-glycolic acid) (PLGA), poly (glycolic acid) (PGA), and mixtures thereof.

The organic polymer may comprise, or consist of, a thermoplastic, such as polylactic acid (PLA), polyhydroxyalkanoate (PHA), polyethylene, or polypropylene, or mixtures thereof.

As an example, poly-lactic acid (PLA) is a widely used biomaterial that has proven its value in various medical applications. PLA is a biodegradable and bioactive thermoplastic aliphatic polyester derived from nature. The sources of PLA are corn starch, cassava roots, chips or starch, or sugarcane. PLA is biodegradable both in nature and in physiological conditions, by simple hydrolysis of the esterbackbone, to form non-harmful and non-toxic compounds. The advantages of PLA in bone engineering are its biocompatibility, thermoplastic and mechanical properties. PLA possesses mechanical properties that make the material suitable for transient load-bearing applications and is easily processed by 3D FDM printing technology. PLA and its copolymers have been used extensively in different fields such as polymer engineering, tissue engineering, drug delivery systems and various medical implants of paramount significance.

PLA can generally exist as poly (L-lactic acid) (PLLA), as poly (D-lactic acid) (PDLA) or as a mixture of PLLA and PDLA, referred to as poly (DL-lactic acid) (PDLLA). The mixture can conveniently be racemic, i.e. a 1:1 mixture of PLLA and PDLA. PDLLA tends to be more amorphous and less crystalline in form than either PLLA and PDLA.

PLA has attracted considerable attention as bone engineering material over the last two decades due to its good processability and properties compared with other biodegradable polymers. PLA with high surface energy has easy printability which makes it widely used in 3D printing. PLA has a glass transition temperature of 60°° C. and a melting temperature at 190° C. (Total Corbion PLA). The basic mechanical properties of PLA are comparable to those of polystyrene and PET. However, the flexural modulus of PLA is higher than polystyrene and PLA has good heat sealability. The PLA compatible with uses according to the invention can be any suitable PLA material, including for example poly-L-Lactide type (PLLA) and poly-D-Lactide (PDLA), including any combinations and mixtures of PLLA and PDLA. In some embodiments, the PLA is PLLA.

Other suitable biomaterials that can also or alternatively be used in the scaffolds and compositions described herein include poly (lactic acid co-glycolic acid) (PLGA), poly (glycolic acid) (PGA), polyhydroxyalkanoate (PHA), polyethylene, or polypropylene, and any mixtures or combinations thereof.

The chitin material can be uniformly mixed in the polymer material. The chitin can be present as particles that are physically dispersed and embedded within the polymer material, i.e. the chitin is present as small distinct particles within the polymer material.

The chitin material can typically can be in the form of microscopic particles that have an average particle diameter in the range of about 0.1 μm to about 50 μm, in the range of about 1 μm to about 25 μm in the range of about 1 to 15 μm or smaller than 20 μm, or in the range of about 5 μm to about 15 μm, or in the range of about 3-10 □m.

It can be appropriate that the tissue scaffold be at least partially porous, i.e. at least parts or portions of the scaffold have a porous structure. By having the scaffold porous, cell recruitment and cell growth into the site of the scaffold is facilitated, thereby enhancing and/or speeding up the tissue regenerative processes at implant site.

Porosity of the scaffold can in general be in the range of about 10% to about 90%, in the range of about 20% to about 70%, in the range of about 30% to about 70%, or the range of about 40% to about about 60%. In some embodiments, the scaffold is about 20% porous, about 25% porous, about 30% porous, about 35% porous, about 40% porous, about 45% porous, about 50% porous, about 55% porous, about 60% porous, about 65% porous, about 70% porous, or about 75% porous. The term “porous”, in this context, refers to the volume of the three-dimensional scaffold structure that is open, i.e. does not contain polymer material.

The porosity and/or the shape of the pores of the implant can be varied as needed, taking into account factors such as the required mechanical strength of the scaffold, the nature of the tissue being in need of regeneration and the amount of chitin material incorportated in the scaffold. In general, the scaffold can have a porosity that is in the range of about 10% to about 90%. The scaffold will have a mechanical strength that can be varied by varying the porosity of the 3D printed structure. Thus, in general the scaffold can have a mechanical strength with yield point in the range of 1-50 MPa, 2-50 MPa, 5-50 MPa, or 10-40 MPa. In some cases the scaffold has a porosity of 50% or less and a mechanical strength of at least 10 MPa.

The tissue scaffold can preferably lack material that is not biodegradable, i.e. the tissue scaffold is entirely biodegradable, with the result that upon being implanted the tissue scaffold slowly degrades in situ over time. Therefore, the tissue scaffold can preferably not contain metal or alloy components or other components that are not biodegradable.

Without intending to be bound by theory, it is believed that he chitin material implanted within a tissue scaffold is degraded by natural degradation processes in the body, catalyzed by natural chitinases, lysozyme or by other natural degradation processes. The degradation process releases short chain chitooligosaccharides, especially with controlled distribution of glucosamine, which are believed to represent the bioactive form of the chitin material, having osteoinductive properties as further described herein.

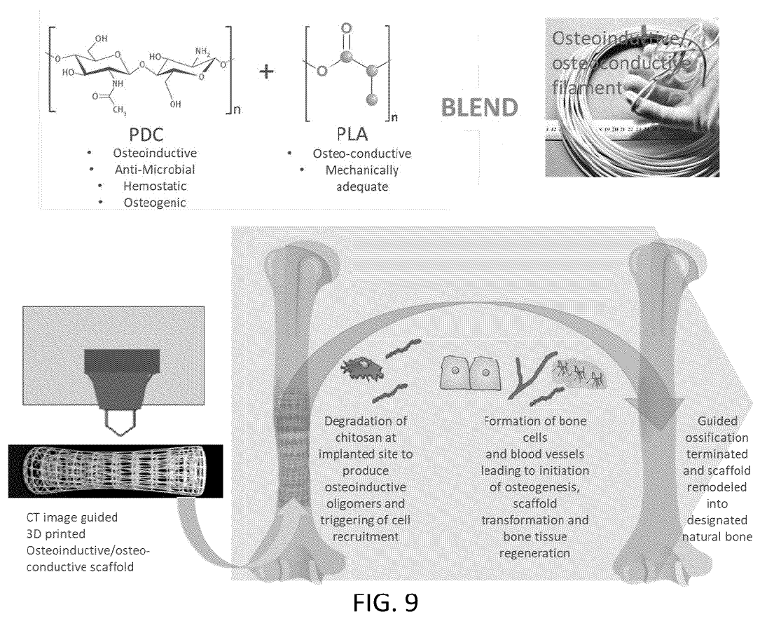

The thus generated chitooligosaccharides trigger tissure regenerative process at the site of implant, for example recruitment of bone forming cells around a 3D printed implant which inserted at a site of a bone defect, together with blood cell formation around the implant site, leading to osteogenic activiation and bone tissue regeneration. With time, the implant, together with the incorporated chitin material, will be fully degraded, but having fulfilled its role of promoting healthy tissue formation. In the case of bone regeneration this includes guiding ossification leading to the tissue remodeling and formation of natural bone tissue. In the context of bone regeneration processes, the conceptual framework in accordance with the present invention can be illustrated as shown in FIG. 9. Thus, the chitin material (exemplified as partially deacetylated chitin or chitosan) has osteoinductive properties, in addition to its known antimicrobial and hemostatic properties. The chitin material can be incorporated in a biocompatible polymer, here exemplified by PLA, from which filaments suitable for 3D printing applications are generated. The filaments are subsequently 3D printed, guided by images obtained by e.g. CT or MRI, to generate a scaffold with bioinductive, e.g. osteoinductive/osteoconductive properties. The thus generated scaffold can then be implanted at a site in need of tissue regeneration, for example a site of a bone defect (e.g., missing bone or bone deformation) including bone fracture. Once implanted, chitin in the scaffold is degraded to release bioactive chitooligosaccharides that promote tissue regeneration at the site of implant, here illustrated by the initiation of osteogenesis and subsequent bone tissue regeneration. With time, the guided ossification process takes over, with the originally implanted scaffold having been degraded by natural processes, and replaced by remodeled natural bone tissue.

The biocompatible polymer can be any suitable polymeric material that provides the required mechanical stability and chemical integrity during 3D printing. Preferably, the biocompatible polymer is biodegradable. Examplary suitable biocompatible polymer materials include polylactic acid (PLA) and polyhydroxyalkanoate (PHA).

It can be advantageous to incorporate calcium phosphate into the biopolymer composite, in addition to chitin. The resulting scaffold will accordingly comprise biopolymer/calcium phosphate composite, within which the chitin material is embedded. Calcium phosphate is a well known bioactive and biodegradable grafting material that is known to be used in bone cement applications. The material can be in the form of crystals, where its crystallinity (crystal size, crystal perfection, grain size) can be varied.

The amount of calcium phosphate can be in the range of 0.2%-20% (w/w), such as in the range of about 0.5%-15% (w/w), in the range of about 0.5%-10% (w/w), in the range of about 1%-10% (w/w), in the range of about 2%-10% (w/w), or in the range of about 2%-8% (w/w).

The calcium phosphate can be in the form of microparticles that can have an average diameter in the range of about 1 to 100 μm, in the range of about 5 to 70 μm, in the range of about 5 to 60 μm, in the range of about 10 to 70 μm, in the range of about 10 to 60 μm, in the range of about 10 to 50 μm, or in the range of about 10 to 40 μm wherein the microparticles are dispersed within the biocompatible organic polymer.

In some embodiments, the calcium phosphate microparticles have a diameter that is less than 80 μm, less than 70 μm, less than 60 μm, or less than 50 μm. In some embodiments, the calcium phosphate microparticles have a diameter that is greater than 2 μm, greater than 5μm, or greater than 10 μm.

The incorporation of calcium phosphate in the composite material described can augment the benefits of chitosan, thereby providing a biopolymer composite with optimal properties (mechanical strength and bioactivity). Thus, by varying the composition (type of biodegradable polymer and the amount of chitosan, with the optional addition of calcium phosphate), the mechanical strength and bioactivity of the resulting composite material can be adapted to the required properties in vivo, with respect to mechanical stability and bioactivity.

Computerized images, for example images obtained by computerized tomography (CT) and magnetic resonance imaging (MRI) can be used to design protocols for 3D printing suitable porous implant scaffolds, for example scaffolds replacing missing and/or fractured bone or bone parts. Thereby, a personalized application is possible, using imaging of a patient in need of bone regenerative therapy to design and produce customized scaffolds by 3D printing that promote natural formation of healthy normal bone tissue at the site of the implant. The bone or bone part to be healed or repaired using the implantable scaffold can be any suitable human or animal bone. For example, the bone can be a human hand bone, a human jaw or skull bone, human face bone (such as nasal or temporal bone), human shoulder bone, human patella bone, human sternum bone, human rib bone, human foot bone or any human load bearing bone (such as but not limited to tibia, fibula, femur, sacrum, sternum, vertebrae).

Printing resolution can be quite high, i.e. a resolution of up to ±0.5 mm, ±0.4 mm, ±0.3 mm, ±0.2 mm, or ±0.1 mm is possible. Printing is also quite fast, i.e. on the order of minutes or at most hours, which means that it is feasible to design, print and introduce an implant in a single medical operation. Mechanically, virtually any bone or bone part in the human or animal body can be designed or replaced using the technology, which thereby provides a unique way to replace and regenerate broken or damaged bone tissue.

The invention can be represented by the following exemplary non-limiting aspects and embodiments:
In one aspect, the invention provides a implantable tissue scaffold comprising a mixture of a biocompatible organic polymer and chitin, wherein the chitin is embedded in the biocompatible organic polymer.
In some embodiments the chitin is partially or fully deacetylated chitin (PDC). In some embodiments the tissue scaffold comprises in the range of about 0.05% to 20% (w/w) chitin.
In some embodiments the tissue scaffold comprises in the range of about 0.05% to 10% (w/w), 0.05% to 5% (w/w), 0.05% to 2% (w/w), 0.5% to 2% (w/w) or 1%-2% (w/w) chitin.
In some embodiments the chitin is chitin having a degree of deacetylation in the range of about 2% to about 99%, in the range of about 6 to 90%, in the range from about 10% to about 70%, in the range of about 20% to about 70%, in the range of about 30% to about 70%, in the range of about 40% to about 60%, or in the range of about 45% to about 55%.
In some embodiments the chitin is chitin having a degree of deacetylation in the range of about 35% to about 75%, in the range of about 35% to about 70%, or in the range of about 40% to about 60%, or in the range of about 45% to about 55%.
In some embodiments, the scaffold contains in the range of about 0.1% to about 1.5% chitin with a degree of deacetylation in the range of about 45% to about 55%.
In some embodiments the chitin has a weight-average molecular weight (MW) in the range of 200 Da-2000 kDa, such as in the range of 1 kDa-1000 kDa, in the range of 5 kDa-500 kDa, in the range of 10 kDa-300 kDa or 20 kDa-150 kDa. The chitin can preferably have a molecular weight in the range of about 100 kDa to about 400 kDa, in the range of about 200 kDa to about 400 kDa, in the range of about 250 kDa to about 350 kDa, such as about 300 kDa.
In some embodiments, the chitin can absorb at least 10x, such as at least 15x or at least 20x, of its dry weight of water.
In some embodiments, the chitin material can be characterized by one or more of (i) a degree of deacetylation in the range of 30-70%, (ii) a weight-average molecular weight in the range of 40 kDa-400 kDa, (iii) ability to form a gel upon contact with water, and (iv) complete (100%) solubility in dilute acid.
In some embodiments the chitin is in the form of microparticles that have an average particle diameter in the range of about 0.1 μm to about 50 μm, in the range of about 1 μm to about 25 μm in the range of about 1 to 15 μm or smaller than 20 μm, or in the range of about 5 μm to about 15 μm.
In some embodiments the microparticles are physically embedded and dispersed within the biocompatible organic polymer. In some embodiments the biocompatible organic polymer is selected from polylactic acid (PLA), poly (lactic acid co-glycolic acid) (PLGA), poly (glycolic acid) (PGA), and mixtures thereof.
In some embodiments the biocompatible organic polymer comprises one or more heat-resistant organic polymer.
In some embodiments the biocompatible organic polymer comprises at least one thermoplastic, such as polylactic acid (PLA), polyhydroxyalkanoate (PHA), polyethylene, or polypropylene, or mixtures thereof.
In some embodiments the tissue scaffold has a porosity in the range of about 10% to about 90%, in the range of about 20% to about 70%, in the range of about 30% to about 70%, or the range of about 40% to about about 60%.
In some embodiments the tissue scaffold further comprises calcium phosphate in an amount in the range of 0.2%-20% (w/w). In some embodiments the calcium phosphate is in the form of microparticles with an average diameter in the range of about 1 to 100 μm, in the range of about 10 to 70 μm, in the range of about 5 to 60 μm, wherein the microparticles are dispersed within the biocompatible organic polymer.
In some embodiments the tissue scaffold does not contain a metal or alloy.
Another aspect relates to a composition for 3D printing, the composition comprising in the range of about 75%-99.95% by weight of at least one biocompatible organic polymer and in the range of about 0.05%-5% chitin, wherein the chitin is embedded within the biocompatible organic polymer. In one such embodiment, the chitin is partially or fully deacetylated chitin.
In some embodiments the composition comprises in the range of about 0.05% to 20% chitin, in the range of about 0.05% to 10% chitin, in the range of about 0.05% to 5% chitin, in the range of about 0.05 to 2% chitin, or in the range of about 1%-2% chitin.
In some embodiments the chitin in the composition is partially deacetylated chitin having a degree of deacetylation in the range of in the range of about 0-75%, 6% to about 70%, about 10% to about about 70%, in the range of about 20% to about 70%, in the range of about 30% to about 70%, in the range about 35% to about 65%, in the range of about 40% to about 70%, in the range of about 40% to about 60%, or the range of about 45% to about 55%.
In some embodiments the chitin in the composition is chitin having a degree of deacetylation in the range of about 35% to about 75%, in the range of about 35% to about 70%, or in the range of about 40% to about 60%, or in the range of about 45% to about 55%.
In some embodiments the chitin in the composition has a weight-average molecular weight (MW) in the range of 200 Da-2000 kDa, such as in the range of 1 kDa-1000 kDa, in the range of 5 kDa-500 kDa, or in the range of 10 kDa-300 kDa or 20 kDa-150 kDa.
In some embodiments the chitin in the composition is in the form of microparticles that have an average particle diameter in the range of about 1 μm to about 50 μm, preferably in the range of about 1 μm to about 25 μm, in the range of about 1 to 15 μm, or in the range of about 5 μm to about 15 μm. In some embodiments the microparticles are dispersed within the biocompatible organic polymer.
In some embodiments the biocompatible organic polymer in the composition is selected from polylactic acid (PLA), poly (lactic acid co-glycolic acid) (PLGA), poly (glycolic acid) (PGA), and mixtures thereof. In some embodiments the biocompatible organic polymer comprises one or more heat-resistant organic polymer.
In some embodiments the biocompatible organic polymer comprises at least one thermoplastic, such as polylactic acid (PLA), polyhydroxyalkanoate (PHA), polyethylene, or polypropylene, or mixtures thereof.
In some embodiments the composition further comprises calcium phosphate in an amount in the range of 0.2%-20% (w/w). In some embodiments the calcium phosphate is in the form of microparticles with an average diameter in the range of about 1 μm to 100 μm, or about 10 μm to 70 μm, or about 5 μm to 60 μm, wherein the microparticles are dispersed within the biocompatible organic polymer.
In some embodiments the density or amount of N-acetylglucosamine (NAG) in the composition is in range of 0.01-100 mg/g, 0.01-50 mg/g, 0.02-15 mg/g, 0.1-15 mg/g, or 0.05-10 mg/g, preferably 0.1-5 mg/g. The amount of NAG in the composition can preferably be calculated per dry weight of material.
Another aspect relates to a method of preparing a tissue scaffold comprising 3D printing a composition as set forth herein.
Another aspect relates to a method of promoting tissue formation, comprising implanting a tissue scaffold as set forth herein at a site in need of regenerative bone tissue formation. In some embodiments the partially deacetylated chitin oligomers comprised in the tissue scaffold are, upon implanting, released from the tissue scaffold in situ, thereby promoting tissue formation.
In some embodiments the tissue is bone tissue.
Another aspect relates to an implantable tissue scaffold as set forth herein for use in the treatment of a bone defect in a human or animal body.

As used herein, including in the embodiments, singular forms of terms are to be construed as also including the plural form and vice versa, unless the context indicates otherwise. Thus, it should be noted that as used herein, the singular forms “a,” “an,” and “the” include plural references unless the context clearly dictates otherwise.

Throughout the description and embodiments, the terms “comprise”, “including”, “having”, and “contain” and their variations should be understood as meaning “including but not limited to”, and are not intended to exclude other components.

The present invention also covers the exact terms, features, values and ranges etc. in case these terms, features, values and ranges etc. are used in conjunction with terms such as about, around, generally, substantially, essentially, at least etc. (i.e., “about 3” shall also cover exactly 3 or “substantially constant” shall also cover exactly constant).

The term “at least one” should be understood as meaning “one or more”, and therefore includes both embodiments that include one or multiple components. Furthermore, dependent embodiments that refer to independent embodiments that describe features with “at least one” have the same meaning, both when the feature is referred to as “the” and “the at least one”.

It will be appreciated that variations to the foregoing embodiments of the invention can be made while still falling within the scope of the invention can be made while still falling within scope of the invention. Features disclosed in the specification, unless stated otherwise, can be replaced by alternative features serving the same, equivalent or similar purpose. Thus, unless stated otherwise, each feature disclosed represents one example of a generic series of equivalent or similar features.

Use of exemplary language, such as “for instance”, “such as”, “for example” and the like, is merely intended to better illustrate the invention and does not indicate a limitation on the scope of the invention unless so embodimented. Any steps described in the specification may be performed in any order or simultaneously, unless the context clearly indicates otherwise. All of the features and/or steps disclosed in the specification can be combined in any combination, except for combinations where at least some of the features and/or steps are mutually exclusive. In particular, preferred features of the invention are applicable to all aspects of the invention and may be used in any combination.

The invention is further described by the following non-limiting examples.

### EXAMPLES

### Example 1

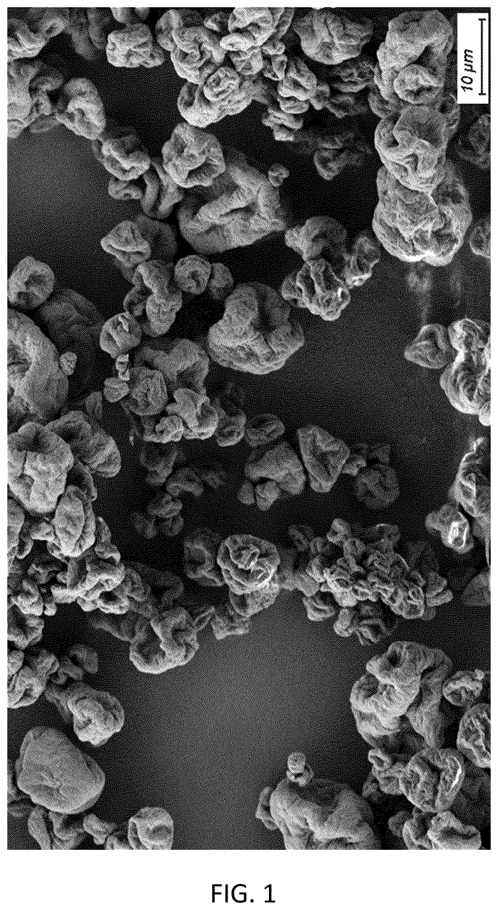

Partially deacetylated chitin (PDC, 50% DD, average MW about 300 kDa) used in the formation of biodegradable composite material was analyzed by scanning electron microscopy (SEM). As can be seen in FIG. 1, the PDC has the appearance of generally ball-shaped particles that have a diameter in the range of about 2 to 10 m.

### Example 2

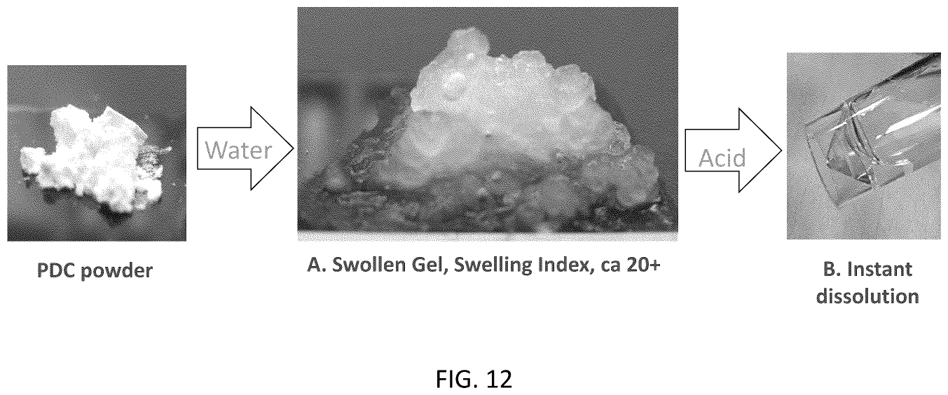

Weigh 50 mg of PDC (50% DD, average MW about 300 kDa) on a small piece of glass plate and apply 3 g of DI water onto its surroundings. Then guide the DI water with a spatula to contact with the PDC to allow the aborption to start. Remove all untrapped water with a piece of filter paper 30 minutes later and record the weight. The PDC transformed from its powdery form into a gel and with a weight of 1010 mg or more than 20X to its original weight (FIG. 12A). Then 50 mg of acetic acid was added to this swollen gel and subsequently stirred with a spatula, the gel transformed into clear solution in seconds (FIG. 12B). This experiment shows that the PDC material can absob more than 20X its dry weight of water and that the material can be completely solubilized in an acidic environment.

### Example 3

Filaments for 3D printing were generated using partially deacetylated chitin (PDC) and the biopolymer polylactic acid (PLA), at several different concentration of PDC in the material as shown in the following Table 1:

TABLE 1

| Materials | PDC, g | PLA, g | Abbreviation |
|---|---|---|---|
| 0.25% PDC/PLA | 0.25 | 100 | 0.25% CS/PLA |
| 0.5% PDC/PLA | 0.50 | 100 | 0.5% CS/PLA |
| 1.5% PDC/PLA | 1.50 | 100 | 1.5% CS/PLA |

Filaments that also include calcium phosphate (CaP) were generated as shown in the below Table 2:

TABLE 2

| Materials | PDC, g | CaPs, g | PLA, g | Abbreviation |
|---|---|---|---|---|
| 0.25% PDC/CaP/PLA | 0.25 | 0.5 | 100 | 0.25% BCCS/PLA |
| 0.5% PDC/CaP/PLA | 0.50 | 1.0 | 100 | 0.5% BCCS/PLA |
| 1.5% PDC/CaP/PLA | 1.50 | 4.5 | 100 | 1.5% BCCS/PLA |

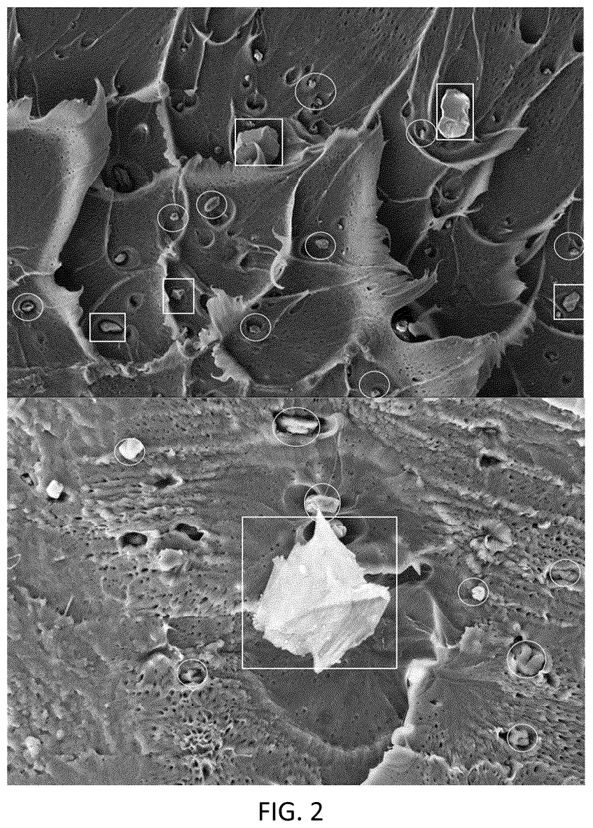

Filaments containing PDC and CaP were analyzed by SEM to visualize the incorporation of PDC. As can be seen in FIG. 2, the filaments contain PDC particles (shown in circles) and larger CaP particles (shown in rectangular boxes).

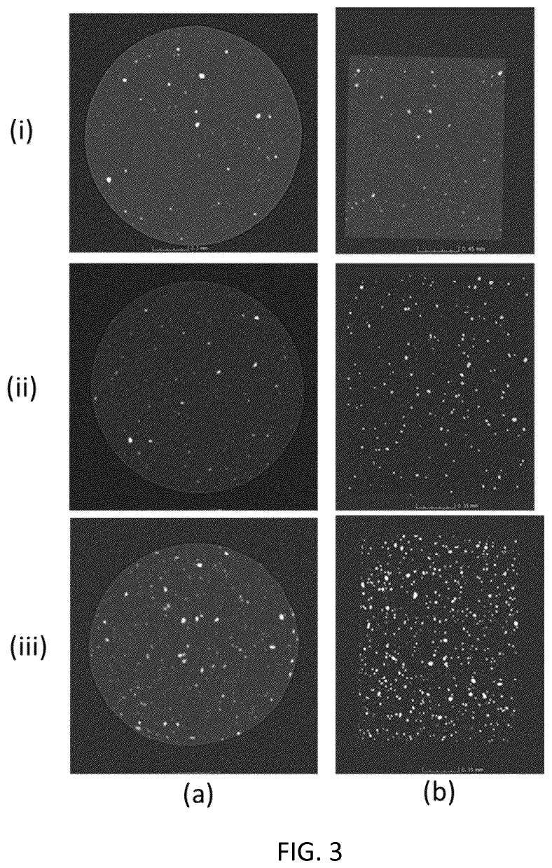

Imaging by micro-CT (FIG. 3) shows the even distribution of CaP particles within the filaments, with cross-sectional views being shown in (a) and longitudinal views in (b) for different composition of CaP/PDC (see Table 2).

### Example 4

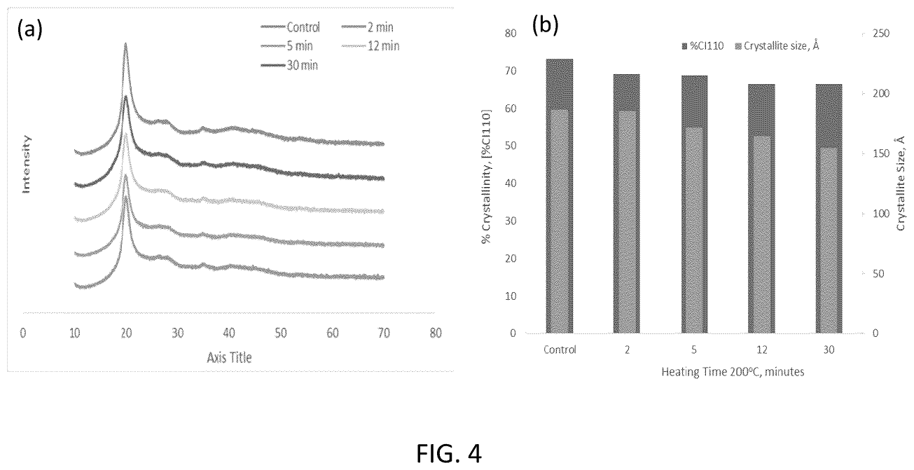

The stability of PDC during 3D printing at 200°° C. was determined by X-ray diffraction (XRD). Results are shown in FIG. 4. As can be seen in (a), all essential peaks for PDC, including reflection at (020), (110) and (130) are intact even after treatment for 30 min at 200° C. This illustrates the stability of PDC upon heat treatment. In (b) there is shown the crystallinity and crystal size, which both decrease slightly with increased treatment time.

### Example 5

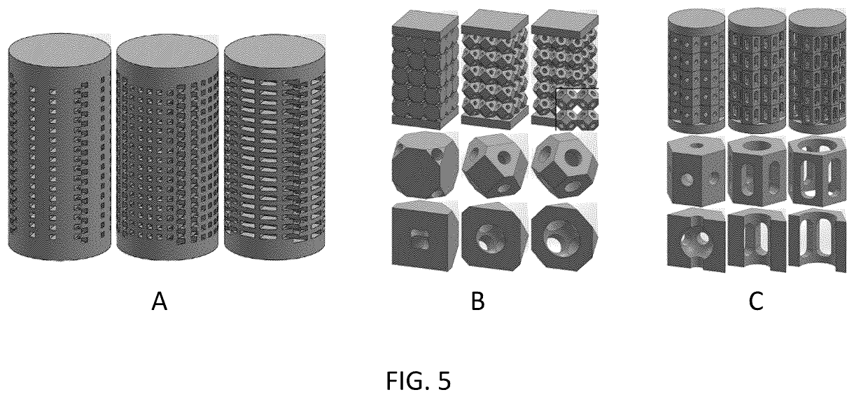

Three types of unit cells were designed for 3D printing of PDC/PLA parts, as illustrated in FIG. 5, including straight stacked beams (SSB, A), porous sodalite crystal (PSC, B) and porous hexagonal prism (PHP, C). Further, the porosity was varied between 25% (a), 50% (b) and 70% (c).

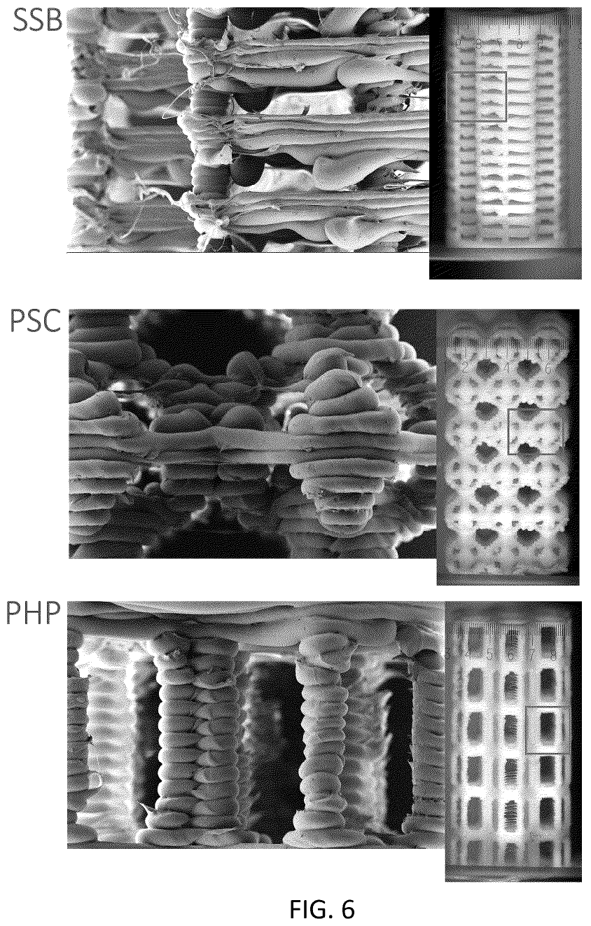

In FIG. 6 scanning electron microscope (SEM) images of printed parts are shown at both macroscopic and microscopic level, with macroscopic views being shown for each cell type on the right, and a microscopic view on the left (within indicated box).

### Example 6

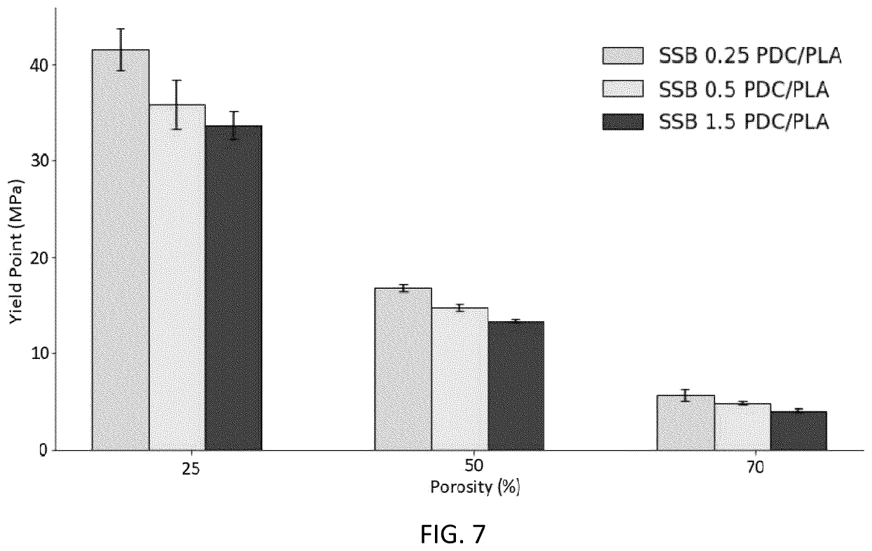

The compressive strength of 3D printed PDC/PLA material was determined, with results as shown in FIG. 7. The compressive strength is highest for the lowest porosity (25%), with a smaller effect being observed as the amound of PDC in the material is increased from 0.25% to 1.5%.

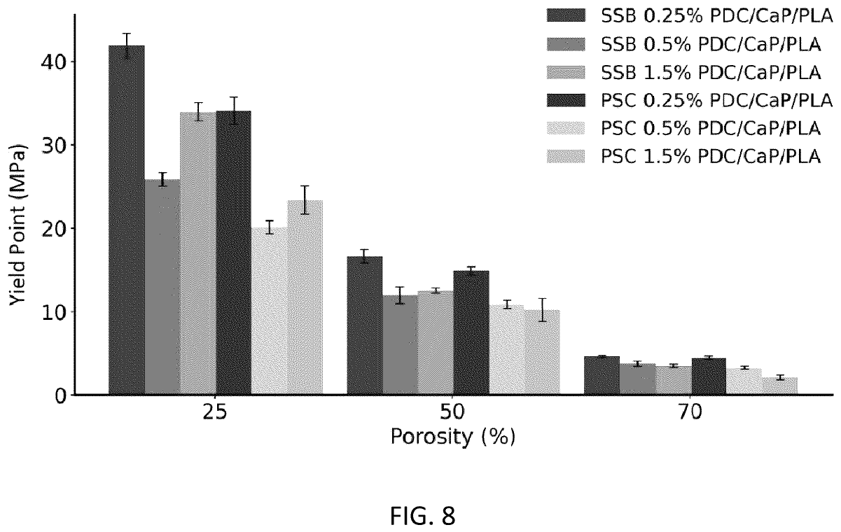

Results for PDC/CaP/PLA material is shown in FIG. 8 for two types of structures, straight stacked beams (SSB) and porous sodalite crystal (PSC). The results indicate that porosity is the greatest determinant of compressive strength of the scaffold.

### Example 7

A clinical trial on 20 rats (Taconic, DK) was performed. The rats were 7-8 months old at the time of operation. Cylindrical 3D printed PLA implants (50% porosity) with an average size of 1×4 mm were inserted into rat femur under general analgesia. The implants contained either no chitosan, or 0.25% or 1.5% chitosan (50% DD, average molecular weight about 300,000 Da). Both femur bones (left; L and right, R) on all the animals received an implant, the experimental protocol being shown in the following table.

MESTABone (7-8 month rats)

| Rat No. | L | R |
|---|---|---|
| 1 | 0.25% CS | Control |
| 2 | 0.25% CS | Control |
| 3 | 0.25% CS | Control |
| 4 | 0.25% CS | Control |
| 5 | 0.25% BCCS | 1.5% BCCS |
| 6 | 0.25% BCCS | 1.5% BCCS |
| 7 | 0.25% BCCS | 1.5% BCCS |
| 8 | 0.25% BCCS | 1.5% BCCS |
| 9 | Control | 0.25% BCCS |
| 10 | Control | 0.25% BCCS |
| 11 | Control | 0.25% BCCS |
| 12 | Control | 0.25% BCCS |
| 13 | 1.5% CS | 0.25% CS |
| 14 | 1.5% CS | 0.25% CS |
| 15 | 1.5% CS | 0.25% CS |
| 16 | 1.5% CS | 0.25% CS |
| 17 | 1.5% BCCS | 1.5% CS |
| 18 | 1.5% BCCS | 1.5% CS |
| 19 | 1.5% BCCS | 1.5% CS |
| 20 | 1.5% BCCS | 1.5% CS |

CS = chitosan (by weight);
BCCS = chitosan plus calcium phosphate, % refers to amount of chitosan by weight

All the animals were healthy in post op monitoring with minimal weight loss. After 3 months, the animals were sacrificed.

Bone formation was assessed using Computerized Tomography (CT), and the volume of bone formation determined.

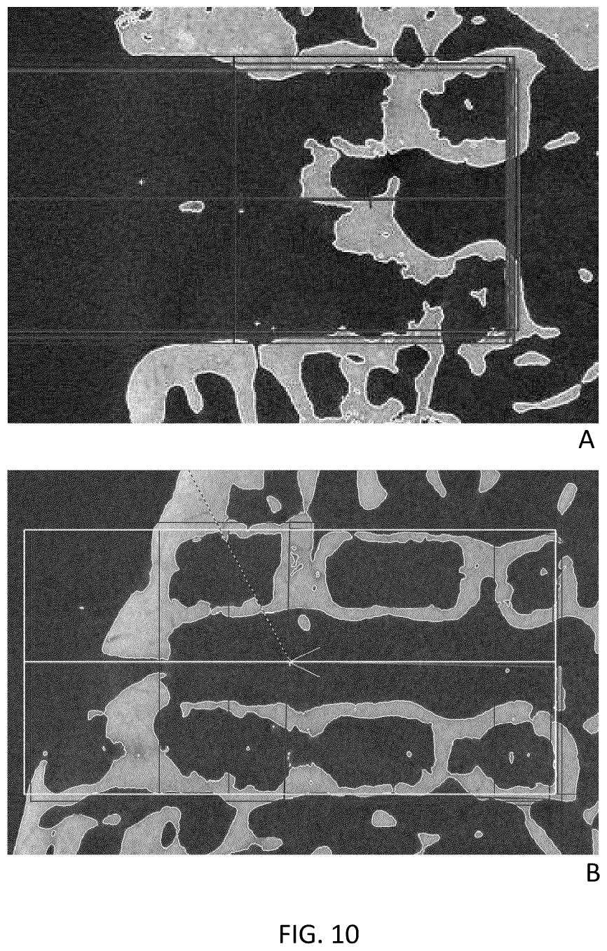

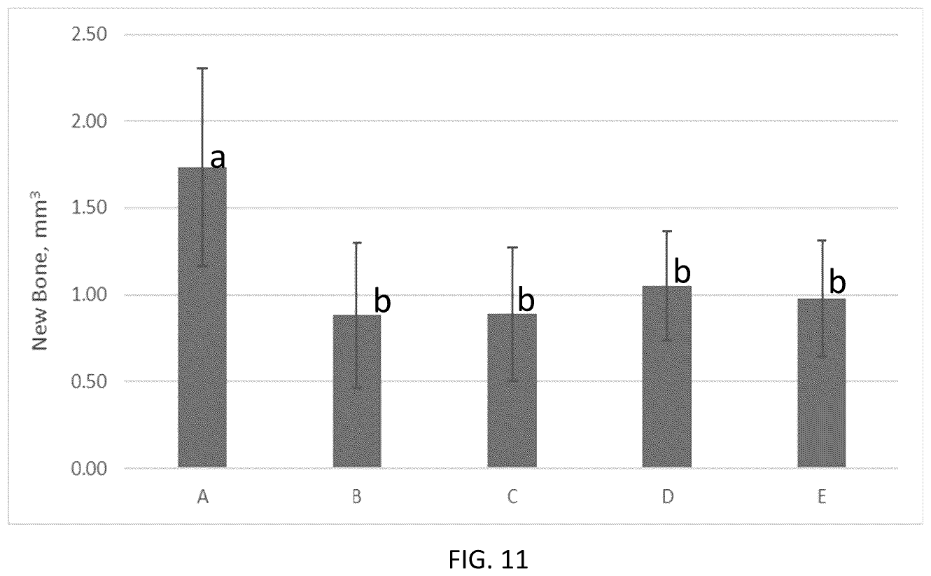

In FIG. 10, representative images of bone formation in animals receiving control implant (PLA implant without chitosan or calcium phosphate) is shown on the left (A), while on the right an image showing bone formation in an animal receiving an implant containing 0.25% chitosan is shown (B). As can be seen, there is extensive bone formation in the animal receiving a chitosan-containing implant, while bone formation in the animal receiving control implant is much less extensive.

In FIG. 11, a comparison of the treatment groups is shown. There is a significant increased formation of new bone (measured as mm³) in implants containing 0.25% compared with control (shown in B). The effect of a higher chitosan content is smaller, although there is a clearly increased bone formation in animals receiving implants containing 1.5% chitosan (D,E) compared with control (B).

Overall, this rat trial shows that even after 3 months, there is a clearly increased bone formation in animals receiving implants containing chitosan compared with control.

1. An implantable tissue scaffold comprising a mixture of a biocompatible organic polymer and chitin, wherein the chitin is embedded in the biocompatible organic polymer, and wherein the tissue scaffold comprises in the range of about 0.05% to 20% (w/w) chitin.

2. The implantable tissue scaffold of claim 1, wherein the chitin is chitin having a degree of deacetylation in the range of about 2% to about 99%, in the range of about 6 to 90%, in the range from about 6% to about 70%, in the range from about 10% to about 70%, in the range of about 20% to about 70%, in the range of about 30% to about 70%, in the range about 35% to about 65%, in the range of about 40% to about 60%, or in the range of about 45% to about 55%.

3. The implantable tissue scaffold of claim 1, wherein the chitin has a random distribution of glucosamine.

4. The implantable tissue scaffold of any one of claims 1, wherein a) the chitin in a dry form is an amorphous chitin; and/or b) wherein the chitin in dry form can absorb 10X or more, 15X or more or 20X or more of its weight of water.

5. (canceled)

6. The implantable tissue scaffold of claim 4b), wherein the chitin, following exposure to water to form a gel, can be dissolved by addition of acid.

7. The implantable tissue scaffold of claim 1, wherein the chitin has a weight-average molecular weight (MW) in the range of 200 Da-2000 kDa, such as in the range of 1 kDa-1000 kDa, in the range of 5 kDa-500 kDa, in the range of 10 kDa-400 kDa, in the range of 20 kDa-400 kDa or in the range of 100 kDa-400 kDa; and/or wherein the chitin is in the form of microparticles that have an average particle diameter in the range of about 0.1 μm to about 50 μm, in the range of about 1 μm to about 25 μm in the range of about 1 to 15 μm or smaller than 20 μm, or in the range of about 5 μm to about 15 μm.

8. (canceled)

9. The implantable tissue scaffold of claim 1, wherein the biocompatible organic polymer is selected from polylactic acid (PLA), poly (lactic acid co-glycolic acid) (PLGA), poly (glycolic acid) (PGA), and mixtures thereof;.

10. The implantable tissue scaffold of claim 1, further comprising calcium phosphate in an amount in the range of 0.2%-20% (w/w), wherein the calcium phosphate is in the form of microparticles that are dispersed within the biocompatible organic polymer, the microparticles having an average diameter in the range of about 1 to 100 μm, in the range of about 10 to 70 μm, or in the range of about 5 to 60 μm.

11. A composition for 3D printing, the composition comprising in the range of about 75%-99.95% by weight of at least one biocompatible organic polymer and in the range of about 0.05%-20% chitin, wherein the chitin is embedded within the biocompatible organic polymer.

12. The composition of claim 11, wherein the chitin is chitin having a degree of deacetylation in the range of about 35% to about 75%, in the range of about 35% to about 70%, or in the range of about 40% to about 60%, or in the range of about 45% to about 55%.

13. The composition of claim 11, wherein the composition contains in the range of 0.01-25 mg/g, or 0.02-15 mg/g, or 0.1-15 mg/g, or 0.05-7.5 mg/g, preferably 0.1-5 mg/g of N-acetylglucosamine (NAG).

14. The composition of claim 11, wherein the chitin has a random distribution of glucosamine.

15. The composition of claim 11, wherein the chitin in a dry form is an amorphous chitin.

16. The composition of claim 11, wherein the chitin in dry form can absorb 10X or more, 15X or more or 20X or more of its weight of water.

17. The composition of claim 16, wherein chitin, following exposure to water to form a gel, can be dissolved by addition of acid.

18. The composition of claim 11, wherein the chitin has a weight-average molecular weight (MW) in the range of 200 Da-2000 kDa, such as in the range of 1 kDa-1000 kDa, in the range of 5 kDa-500 kDa, or in the range of 10 kDa-300 kDa or in the range of 20 kDa-300 kDa; and/or wherein the chitin is in the form of microparticles that have an average particle diameter in the range of about 1 μm to about 50 μm, preferably in the range of about 1 μm to about 25 μm, in the range of about 1 to 15 μm, or in the range of about 5 μm to about 15 μm.

19. (canceled)

20. The composition of claim 11, wherein the biocompatible organic polymer is selected from polylactic acid (PLA), poly (lactic acid co-glycolic acid) (PLGA), poly (glycolic acid) (PGA), and mixtures thereof.

21. The composition of claim 11, further comprising calcium phosphate in an amount in the range of 0.2%-20% (w/w), wherein the calcium phosphate is in the form of microparticles that are dispersed within the biocompatible organic polymer, the microparticles having an average diameter in the range of about 1 to 100 μm, in the range of about 10 to 70 μm, or in the range of about 5 to 60 μm.

22. A method of preparing a tissue scaffold, the method comprising 3D printing a composition as set forth in claim 11.

23. An implantable tissue scaffold according to claim 1 for use in the treatment of a bone defect in a human or animal body.
